(19) United States
(12) Patent Application Publication
(10) Pub. No.: US 2007/0254026 A1
(43) Pub. Date: Nov. 1, 2007

(54) SOFTGEL CAPSULES

(75) Inventor: David John Stewart, North Vancouver (CA)

Correspondence Address: KIRTON AND MCCONKIE, 60 EAST SOUTH TEMPLE,, SUITE 1800, SALT LAKE CITY, UT, 84111, US

(73) Assignee: FORBES MEDI-TECH INC., Vancouver (CA)

(21) Appl. No.: 11/743,091

(22) Filed: May 1, 2007

Related U.S. Application Data
(60) Provisional application No. 60/796,990, filed on May 1, 2006.

(51) Int. Cl. A61K 9/64; A61K 31/56; A61K 36/889; A61K 36/899; A61K 36/48; A61K 36/31; A61K 36/55; A61K 36/42

(52) U.S. Cl. 424/456; 424/727; 424/755; 424/750; 424/757; 424/758; 424/768; 424/769; 514/171

(57) ABSTRACT

A composition for use in softgel capsules comprises one or more esters of phytosterols and/or phytostanols which have been treated to enhance their flowability at ambient temperatures prior to or concurrent with softgel formation.

### FIELD OF THE INVENTION

This present invention relates to the field of nutraceuticals, particularly softgel capsules, comprising phytotserol and phytostanol esters.

### BACKGROUND OF THE INVENTION

While recent advances in science and technology are helping to improve quality and add years to human life, the prevention of atherosclerosis, the underlying cause of cardiovascular disease (“CVD”) has not been sufficiently addressed. Atherosclerosis is a degenerative process resulting from an interplay of inherited (genetic) factors and environmental factors such as diet and lifestyle. Research to date suggest that cholesterol may play a role in atherosclerosis by forming atherosclerotic plaques in blood vessels, ultimately cutting off blood supply to the heart muscle or alternatively to the brain or limbs, depending on the location of the plaque in the arterial tree¹,². Data from the early Framingham Epidemiological Study indicates that increases in serum cholesterol levels are associated with increased risk of death from CVD³. More recent studies confirm that CVD is a leading cause of death and disability in industrialized nations⁴.

Studies have indicated that a 1% reduction in a person's total serum cholesterol yields a 2% reduction in risk of a coronary artery event⁵. Statistically, a 10% decrease in average serum cholesterol (e.g. from 6.0 mmol/L to 5.3 mmol/L) may result in the prevention of 100,000 deaths in the United States annually⁶.

As the population becomes increasingly aware of the importance of maintaining cholesterol balance in check, the need for naturally derived, safe and effective agents which address the underlying causes of CVD, and which can be readily incorporated into a wide variety of delivery means, becomes even more apparent.

One focus of such research related to naturally derived, safe and effective agents to address the underlying causes of CVD has been plant-derived sterols and stanols (also known as phytosterols and phytostanols). Sterols are naturally occurring compounds that perform many critical cellular functions. Phytosterols such as campesterol, stigmasterol and beta-sitosterol in plants, ergosterol in fungi and cholesterol in animals are each primary components of cellular and sub-cellular membranes in their respective cell types. The dietary source of phytosterols in humans comes from plant materials i.e. vegetables and plant oils. The estimated daily phytosterol content in the conventional western-type diet is approximately 60-80 milligrams in contrast to a vegetarian diet which would provide about 500 milligrams per day.

Phytosterols have received a great deal of attention due to their ability to decrease serum cholesterol levels when fed to a number of mammalian species, including humans. While the precise mechanism of action remains largely unknown, the relationship between cholesterol and phytosterols is apparently due in part to the similarities between the respective chemical structures (the differences occurring in the side chains of the molecules). It is assumed that phytosterols displace cholesterol from the micellar phase and thereby reduce its absorption or possibly compete with receptor and/or carrier sites in the cholesterol absorption process.

Over forty years ago, Eli Lilly marketed a sterol preparation from tall oil and later from soybean oil called Cytellin™ which was found to lower serum cholesterol by about 9% according to one report.⁷ Various subsequent researchers have explored the effects of sitosterol preparations on plasma lipid and lipoprotein concentrations⁸ and the effects of sitosterol and campesterol from soybean and tall oil sources on serum cholesterols.⁹ Compositions have been explored in which phytosterols or phytostanols (their hydrogenated counterparts) are esterified in order to enhance solubility. One composition of phytosterols which has been found to be highly effective in lowering serum cholesterol is disclosed in U.S. Pat. No. 5,770,749 to Kutney et al.

Despite the obvious and now well recorded advantages of phytosterols, not only in the treatment of CVD and its underlying conditions such as hypercholesterolemia, hyperlipidemia, atherosclerosis, hypertension, thrombosis but in the treatment of other diseases such as Type II diabetes, dementia cancer and aging, the administration of phytosterols and the incorporation thereof into foods, pharmaceuticals and other delivery vehicles has been complicated by the fact that they are highly hydrophobic (i.e. they have poor water solubility). This highly hydrophobic nature of phytosterols renders them insoluble and barely dispersible in aqueous media. As such, phytosterols tend to be added to the fat phase of fat-based food products. Health-conscious consumers wishing to benefit from the cholesterol lowering effects of phytosterols are therefore forced to consume fat- rich foods, despite the health risks of a high fat diet.

In addition, and critically in the area of food and beverage production, free, unesterified phytosterols have a waxy consistency and a high melting point, creating solubility issues for the food processor. While they are oil-dispersible to some extent in their raw form, the amount required to produce an efficacious effect in a finished product can cause granulation.

The current authors attempted to develop a softgel capsule comprising a composition of phytosterols/stanols omega-3-polyunsaturated fatty acids. One difficulty that was encountered was that mixing free sterols/stanols with vegetable oil resulted in a mixture that quickly thickened into a cement-like material over a few hours that could not be encapsulated. Adding a higher proportion of oil to sterols/stanols plus suspending agents overcame that problem but created a serious stability problem. The sterols within the capsule, over a period of 3-6 months at room temperature, formed a hard shell of sterols underneath the gelatin capsule. On administration, the gelatin shell dissolves quickly but the inner sterol shell remained intact and passes through the digestive tract. Thus, the contents of the sterol shell were biologically unavailable. This problem is referred to as “Ostwald Ripening”.

The current answer to this problem is to esterify the phytosterols/stanols, which creates equilibrium between the phytosterols and liquid oil.

Esterification of phytosterols generally results in lowered melting temperatures. Thus, such phytosterol esters generally may be incorporated into food products more readily and can provide food products without significantly gritty texture. Although the problem of fat solubility of phytosterols can be improved by esterification, this is not a completely satisfactory solution for incorporation into some delivery vehicles, such as soft gelatin (softgel) capsules, as there are additional obstacles in this particular vehicle. Firstly, esters are not liquid or pourable at room temperature. As such, in any process of manufacturing capsules, esters must be heated to above 35-40° C. during or prior to filling into the capsules. In reality, in order to avoid solidification in the encapsulation equipment, the ester temperature needs to be maintained at or above 40-50° C. Phytosterol/stanol esters are usually sold and transported in 180 kg or larger size containers. The time required to bring a 180 kg barrel of esters to 40-50° C. is 3 to 6 days with specific heating equipment being required. There is no commercially reasonable way to remove smaller amounts of the esters from the transport barrel to process in smaller increments. From a manufacturing perspective, this is a significant impediment to softgel capsule formation due to the time and extra handling required.

Secondly, phytosterol/stanol esters are highly viscous and are not readily amenable to softgel economic softgel capsule processing.

It is an object of the present invention to obviate or mitigate the above noted disadvantages.

### SUMMARY OF THE INVENTION

The present invention provides a composition for use in softgel capsules comprising one or more esters of phytosterols and/or phytostanols which have been treated to enhance their flowability at ambient temperatures prior to or concurrent with softgel formation.

The present invention provides a composition for use in softgel capsules comprising one or more esters of phytosterols and/or phytostanols which have been treated to reduce their viscosity at ambient temperatures.

The present invention provides a composition for use in softgel capsules comprising one or more esters of phytosterols and/or phytostanols which have been pre-mixed with an edible oil prior to softgel capsule formation in order to enhance the flowability of the esters at ambient temperatures.

The present invention provides a composition for use in softgel capsules comprising one or more esters of phytosterols and/or phytostanols which have been pre-mixed with an edible oil comprising omega polyunsaturated fatty acids prior to softgel capsule formation in order to reduce the viscosity of the esters at ambient temperatures.

The present invention further provides softgel capsules comprising one or more esters of phytosterols and/or phytostanols which have been treated to enhance their flowability at ambient temperatures prior to or concurrent with softgel formation.

The present invention further provides a method of stabilizing from oxidation a composition of one or more esterified phytosterols and phytostanols such composition being useful for softgel capsule filler, which comprises solubilizing in the esters one or more free (unesterified) phytosterols or phytostanols.

The present invention further provides a method of stabilizing from oxidation a composition of one or more esterified phytosterols and phytostanols such composition being useful for softgel capsule filler, which comprises mixing the ester with an edible oil into which has been solubilised one or more free (unesterified) phytosterols or phytostanols.

Surprisingly, it has been found that the formation of softgel capsules comprising phytosterol and/or phytostanol esters is greatly facilitated by one or both of the following:
1) pre-mixing the esters with even a very small amount of an edible oil; and/or
2) solubilizing within the esters even a very small amount of a free (unesterified) sterol and/or stanol

The edible oil, preferably as described below, is high in omega polyunsaturated fatty acids, decreases the viscosity of the esters and enhances their handling and pourability. The free sterols/stanols, solubilised within the ester, have been found to decrease the oxidation of the esters thereby increasing the useful life the capsule. In the alternative, free sterols/stanols are solubilized in an edible oil, and then mixed with the ester forming a composition suitable for softgel filler.

The free, unesterifed phytosterols and phytostanols (preferably phytostanols) can readily be dissolved in phytosterol and/or phytostanol esters by heating the mixture to approximately 90° C. The free phytosterols/stanols remain dissolved once the mixture is cooled. This dissolution in the esters presents a number of advantages. One is that the presence of the free moiety (stanols and sterols) stabilizes from oxidation and rancidity the ester moiety and as such the compositions remain useful for longer post-manufacturing as filler for and within the softgel capsules. The edible oil also reduces the melting point of the esters, so that the composition becomes fluid at room temperature, eliminating the need for special warming equipment thereby saving significantly on softgel capsule manufacturing costs. This is a critical advantage which not only assists in manufacturing but reduces materials loss, due to room temperature flowability.

The softgel capsules of the present invention have an enormous number of therapeutic uses when administered to animals, in particular humans, not only in respect to the treatment of cardiovascular disease and its underlying conditions such as hypercholesterolemia, hyperlipidemia, atherosclerosis, hypertension, thrombosis but in the treatment and inhibition of other diseases such as Type II diabetes, dementia (including Alzheimer's disease), neural degeneration, cancer (including colon and prostate), and mental disorders such as bipolar disease. In addition, the softgel capsules may be used to enhance brain development and visual acuity.

These effects and other significant advantages will become apparent herein below.

### PREFERRED EMBODIMENTS OF THE INVENTION

The following detailed description is provided to aid those skilled in the art in practising the present invention. However, this detailed description should not be construed so as to unduly limit the scope of the present invention. Modifications and variations to the embodiments discussed herein may be made by those with ordinary skill in the art without departing from the spirit or scope of the present invention. Unless otherwise defined, all technical and scientific terms used herein have the same meaning as commonly understood by one of ordinary skill in the art to which this invention pertains.

As used herein, “animal” means any member of the animal kingdom, including preferably humans.

As used herein, the term “phytosterol” includes all sterols without limitation, for example: sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol, and all natural or synthesized forms and derivatives thereof, including isomers. The term “phytostanol” refers to saturated or hydrogenated sterols including all natural or synthesized forms and derivatives thereof, and isomers. It is to be understood that modifications to the phytosterols and phtostanols i.e. to include side chains also falls within the purview of this invention. For example, the purview of this invention clearly includes 24 beta-ethylsitostanol, 24-alpha-ethyl-22-dehydrositostanol. It is also to be understood that, when in doubt throughout the specification, and unless otherwise specified, the term “phytosterol” encompasses both sterol and stanol. In a most preferred form, the sterol is in its saturated form and is a sitostanol, preferably beta-sitostanol.

These sterols and stanols for use in accordance with this invention may be procured from a variety of natural sources. For example, they may be obtained from the processing of plant oils (including aquatic plants) such as corn oil and other vegetable oils, wheat germ oil, soy extract, rice extract, rice bran, rapeseed oil, sunflower oil, sesame oil and fish (and other marine-source) oils. They may also be derived from fungi, for example ergosterol. Accordingly, the present invention is not to be limited to any one source of sterols. U.S. Pat. No. 4,420,427 teaches the preparation of sterols from vegetable oil sludge using solvents such as methanol. Alternatively, phytosterols and phytostanols may be obtained from tall oil pitch or soap, by-products of forestry practises as described, for example, in U.S. Pat. No. 5,770,749, incorporated herein by reference.

Phytosterols and phytostanols, as used herein, may be in their free form or in one or more of their esterified forms i.e. optionally, the phytosterol or phytostanol may be esterified prior to formation of the food products. This esterification step renders the phytosterols and/or phytostanols more soluble in fats and oils which may, in some instances, facilitate the incorporation of the phytosterols into various food products.

To form phytosterol and/or phytostanol esters, many methods are known in the art. For example, one or more suitable aliphatic acids or their esters with low boiling alcohols may be condensed with the selected phytosterol and/or phytostanol. A wide variety of aliphatic acids or their esters may be used successfully and include all aliphatic acids consisting of one or more alkyl chains with one or more terminal carboxyl groups. These aliphatic acids may be natural of synthetic and are represented by the following chemical formulae:

a) R1-COOH (monocarboxylic acid) wherein:
R1 is an unbranched saturated alkyl group, represented by CH₃—, CH₃CH₂— or
CH₃(CH₂)ₙCH₂— WHERE n=3-25; or
R1 is a branched saturated alkyl group represented by CₙH₂ₙ₊₁ where n=1-25 is the number of carbon atoms contained in the group R1; the branching typically refers, but is not limited to one or more methyl group side chains (branches); or
R1 is an unbranched or branched unsaturated alkyl group, represented by the formula CₙH₂ₙ₋₂ₘ₊₁, where n=1-25 is the number of carbon atoms in R1 and
m=degree of unsaturation; or
   b) HOOC—R2-COOH is a dicarboxylic acid wherein:
R2 is an unbranched saturated alkyl group, represented by —CH₂—, or —CH₂CH₂—, or —CH₂(CH₂)ₙCH₂ where n=3-25; or
R2 is a branched saturated alkyl group represented by —CₙH₂ₙ— where n=1-25 is the number of carbon atoms contained in the group R2; the branching typically refers, but is not limited to, one or more methyl group side chains (branches); or
R2 is an unbranched or branched unsaturated alkyl group, represented by the formula CₙH₂ₙ₋₂ₘ, where n=1-25 is the number of carbon atoms in R2 and
m=degree of unsaturation; or
   c) a tricarboxylic acid represented by the formula:

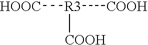

wherein, in this formula:
R3 is a branched saturated alkyl group represented by —CₙH₂ₙ₋₁— where n=1-25 is the number of carbon atoms contained in the group R3;
the branching typically refers, but is not limited to, one or more methyl group side chains (branches); or
R3 is a branched unsaturated alkyl group, represented by CₙH₂ₙ₋₂ₘ₋₁ wherein n=1-25 is the number of carbon atoms in R3 and m=the degree of unsaturation; or
   d) a mono-. di-, or tricarboxylic acid as defined above, which may contain one, two or three hydroxyl groups in the molecule.

In a preferred form, the acid is either a straight-chain or branched unsaturated or saturated, aliphatic or aromatic acid. More preferably, the acids are selected, inter alia, from the following list: valeric acid, isovaleric acid, sorbic acid, isocaproic acid, lauric acid, myrestic acid, palmitic acid, stearic acid, caproic acid, ascorbic acid, arachidic acid, behenic acid, hexacosanoic acid, octacosanoic acid, pentadecanoic acid, erucic acid, linoleic acid, linolenic acid, arachidonic acid, acetic acid, citric acid, tartaric acid, palmitoleic acid and oleic acid. The most preferable fatty acids within the scope of the present invention are linoleic acid, linolenic acid and arachidonic acid which may be obtained from natural sources such as safflower oil, sunflower oil, olive oil and corn oil (linoleic acid), safflower oil, sunflower oil, olive oil and jojoba oil (linolenic acid and arachidonic acid) and rapeseed oil (erucic acid). It is fully contemplated within the scope of the present invention the sterol and stanol esters may be formed with fatty acids selected from: eicosapentaenoic acid (EPA), docosahexanoic acid (DHA), and alpha-linolenic acid (ALA).

Other aromatic acids are clearly contemplated within the scope of the present invention.

By way of example, to form a phytosterol ester, the selected phytosterol and acid or its ester with volatile alcohol may be mixed together under reaction conditions to permit condensation of the phytosterol with the acid. A most preferred method of preparing these esters which is widely used in the edible fat and oil industry is described in U.S. Pat. No. 5,502,045 (which is incorporated herein by reference). As no substances other than the free phytosterol, a fatty acid ester or mixture thereof and an interesterification catalyst like sodium ethylate are used, the technique is highly suitable for preparing products ultimately for human consumption. In overview, this preferred method, adapted for use within the present invention, comprises heating the phytosterol(s) with a vegetable oil fatty acid ester (preferably a methyl ester) at a temperature from 90-120° C. and subsequently adding a suitable catalyst such as sodium ethylate. The catalyst is then removed/destroyed by any one of the techniques known in the art e.g. adding water and/or filtration/centrifugation.

Another method which may be used in accordance with the present invention is described in U.S. Pat. Ser. No. 4,588,717, which is also incorporated herein by reference. A preferred method is to mix the phytosterol and the fatty acid together bringing the mixture to a temperature of from about 15° C. to about 45° C. at about atmospheric pressure for approximately one to three hours.

Accordingly, it is to be understood that the widest possible definition is to be accorded to the terms “phytosterol” ester and “phytostanol” ester as used herein, including, but not limited to: esterified phytosterols and phytostanols with aliphatic or aromatic acids (thereby forming aliphatic or aromatic esters, respectively), phenolic acid esters, cinnamate esters, and ferulate esters. It is also to be understood that the term “phytosterols” as used herein, whether singular or plural, unless otherwise indicated, includes both phytosterols and phytostanols.

In one preferred form, the composition of the present invention useful as a “filler” for use in softgel capsules comprises a ratio of omega polyunsaturated fatty acid oil to sterol/stanol esters from about 5:1 to 30:1. In a further preferred form, the composition comprises a ratio of omega polyunsaturated fatty acid oil to sterol/stanol esters from about 1:5 to 1:20. Whether significantly more sterol/stanol ester or omega PUFA oil is present in the capsule depends largely on the therapeutic indication to be treated or prevented. For example, in the treatment of benign prostatic hyperplasia, it is preferred that the capsules comprise significantly more omega polyunsaturated fatty acid oil than esters than would be preferred in capsules for the treatment of cardiovascular disorders. This is apparent from the examples provided hereinbelow.

The present invention includes:
softgel capsules comprising one or more esters of phytosterols and/or phytostanols and component to enhance the flowability of the esters.
softgel capsules comprising one or more esters of phytosterols and/or phytostanols and component to reduce the viscosity of the esters at ambient temperatures.
softgel capsules comprising one or more esters of phytosterols and/or phytostanols and a measurable amount of one or more free phytosterols and/or phytostanols.
softgel capsules comprising one or more esters of phytosterols and/or phytostanols and an edible oil.
softgel capsules comprising one or more esters of phytosterols and/or phytostanols a measurable amount of one or more free phytosterols and/or phytostanols and an edible oil.
Any of the above softgel capsules comprising omega-3, omega-6 and/or omega-9 polyunsaturated fatty acids.

In another preferred form of the invention, the following ratio of ester:free sterols and/or stanols are contemplated:

| Sterol and/or | Free Sterol |
|---|---|
| Stanol Ester (wt %) | and/or Stanol (wt %) |
| 55–99.8 | 0.2–45 |
| 80–99 | 1–20 |
| 90–98 | 2–10 |
| 95–97 | 3–5 |

In a further preferred form of the present invention, the composition comprises from 1-15% by weight unesterified phytosterol and/or phytostanol substantially completely dissolved in 85-99% by weight of one or more esterified phytosterols and/or phytostanols. In a further preferred form, the composition comprises from 4-8% by weight unesterified phytosterol and/or phytostanol substantially completely dissolved in 92-96% by weight of one or more esterified phytosterols and/or phytostanols.

Within a most preferred form of this invention, it was found that if phytosterols/stanols were first converted to an ester with an edible oil, the ester readily co-mixes with omega polyunsaturated fatty acids and forms a low viscosity solution at room temperature which can be easily encapsuled into soft gelatin capsules. These capsules are expected to remain stable for at least one year. This innovation offers a number of advantages besides product stability. As described above, sterol esters only become liquid above 35-40 degrees C. With the composition proposed herein, the esters remain liquid and pourable at ambient temperatures. As such, no special processing equipment is required to form the capsules.

In a preferred form of the present invention the composition for use in forming the softgel capsules comprises an edible oil to which the free (unesterifed) phytosterols and/or phytostanols are added prior to the substantially complete dissolution in the esterified phytosterols and/or phytostanols. The edible oil may be selected from but is not limited to the group consisting of olive, rapeseed, canola, sunflower, safflower, sesame, soyabean, corn, coconut, peanut, cottonseed, hemp, flaxseed, and pumpkinseed. Most preferably, the edible oil selected is high in one or more of omega 3 polyunsaturated fatty acids, omega 6 polyunsaturated fatty acids and omega 9 polyunsaturated fatty acids. Such an addition of edible oil reduces the melting point of the esters, so that the composition becomes and stays fluid at room temperature, thereby completely eliminating the need for special warming equipment during processing of the esters.

### Omega-3 Fatty Acids

There are distinct additional advantages of the incorporation of omega—fatty acids in the capsules. Omegas are known to reduce the build up of atherosclerotic plaque in the arteries (Von Schacky.2000; Erkkila.2004; Renier.1993). The omega fatty acids act by mechanisms independent of the reduction in LDL. They have anti-oxidant activity (Frenoux.2001) and anti-inflammatory activity (Maroon.2006) and can raise HDL (good cholesterol) levels and may contribute to reduction in atherosclerotic plaque formation by these mechanisms. Omega fatty acids also have anti-coagulant properties (Schmidt.2001; Mori.2003).

Conversely, omega-3-fatty acids have the undesirable effect of raising blood LDL in the human (Harris.1996; Theobald.2004). The effect may be related to the anti-oxidant effect of omega-3-fatty acids (Kraus.2004). However, when omega-3-fatty acids are combined with phytosterols, this side effect is eliminated, while still maintaining maximum efficacy of the phytosterols in reducing blood LDL reductions.

In addition, omega fatty acids have additional synergistic and protective effects against cardiovascular disease not provided by phytosterols. Omega fatty acids have been demonstrated to reduce triglycerides (Roche.1996) and blood pressure (Frenoux.2001;Prisco.1998), two additional risk factors for heart disease. In the final stage of a fatal heart attack, damage to the myocardial tissue results in fibrillation. Omega-3-fatty acids have been demonstrated to reduce the risk of dangerous abnormal heart rhythms (Moazffarian.2004), the risk of sudden cardiac death (Nordoy et al 2001; Stone 2000;Marchioli.2002) and obstructive stroke in the elderly (Moazffarian.2005).

The combination within the softgel capsules may have other synergies as well. It is now recognized that lifestyle changes or treatments that reduce the risk of cardiovascular disease also reduce the risk of Alzheimers Disease (Luchsinger.2004). FDA has allowed a health claim for phytosterols “may reduce the risk of coronary heart disease” (FDA#1). The same but qualified claim may also be made for omega-3-fatty acids from a fish source (FDA#2). Tall oil phytosterols (β-sitosterol) have been demonstrated to reduce the symptoms of benign prostate hypertrophy (Berges.2000; Coleman.2002). The effective dose of sitosterol is in the range of 20 to 60 mg per day. Both phytosterols (Awad.2000) and omega-3-fatty acids have been observed to either reduce the risk and/or retard the development of prostate cancer (Awad.2002; Augustsson.2003). There is evidence that both phytosterols and omega fatty acids can reduce the risk and/or retard the development of bowel and breast cancer (Awad.2002; Cayhill.1996).

Both phytosterols and fish source omega-3-fatty acids are well recognized to be beneficial to heart health. In selecting the dosages and ratios of these two components, marketing considerations and the types of health claims that can be made in different jurisdictions may be considered. The actual product configuration requires balancing consumer preferences with product cost and the types of health claims that can be made.

Dose ranging studies with phytosterols demonstrate that a LDL cholesterol reduction is detectible at a dose of 0.8 g/day or higher. The FDA allows a heart health claim to be made for phytosterols at doses of 0.8 g/day or higher (21CFR 101.83). Meta analysis of a large number of studies shows that the effect of phytosterols plateaus at 2 g/day (Katan et al 2003). Examples with high dose phytosterols (2.0 g/day) can be found in Tables 2-7, and 10-11. Examples of medium dose phytosterols (1.0 g g/day) which can qualify for the FDA health claim can be found in Table 8.

For supplements, 2 g/day of fish source omega-3-fatty acids is the top end of the allowed range in the US. The FDA has set the upper limit for supplements at 2 g/day of omega-3-fatty acids (FDA letter of October 2000). Because the effects of omega-3-fatty acids are multiple, the dosage targeted depends on what is being claimed. At the high end of the dosage range, omega-3-fatty acids reduce the blood levels of triglycerides. This has benefit in protecting against pancreatitis associated with certain medications, diabetes, and excessive alcohol intake (Pejic.2006). In the EU, a dose of 1.5 g/day qualifies for the claim “helps control levels of triglycerides”. In Canada, a dose 1.0 g/day qualifies for the claim “helps to reduce serum triglycerides”. Elevated levels of triglycerides contribute to the development of Type 2 diabetes, a major risk factor for heart disease. In large secondary prevention studies, doses of omega-3-fatty acids (0.5 g -1.8 g/day) have been demonstrated to reduce the risk of heart attacks, sudden death, and all-cause mortality. American Heart Association statement (Kris-Etherton, 2002). In Canada, a dose of >0.5g/day qualifies for the claim “helps maintain/support cardiovascular health”. In the EU, a dose of >0.450 g/day of long chain omega-3-polyunsaturated acids qualifies for the claim “helps maintain heart health”. Examples of capsules with 2g/day omega-3-fatty acids (high dose) are found in Tables 2, 3, 1.0 g/day (medium dose)in Tables 4, 5, 8 and 0.5 g/day (low dose) in Tables 6,7.

### Alzheimers Disease: Combination of Phytosterols with Omega-3-Fatty Acids

Alzheimer's disease is the most common form of dementia and currently affects over 13 million people worldwide. The direct and indirect cost of Alheimer's disease care is over $100 billion in the US alone. A number of studies have shown that fish source omega-3-fatty acids can delay cognitive decline in the elderly (Beydoun.2007). The onset of Alzheimers disease is also associated with abnormalities in cholesterol metabolism, elevated cholesterol levels, and atherosclerosis. There is some evidence of a significantly reduced incidence of Alzheimer's disease among people who have been using statins to reduce hypercholesterolaemia and its cardiovascular effects (Whitfield.2006). Similarly, phytosterols by reducing LDL cholesterol levels, may have the same effect. A combination of phytosterols and omega-3-fatty acids is therefore expected to have an added benefit to that of omega-3-fatty acids in delaying the onset of Alzheimer's disease. Tables 2-5 provide examples of formulations with high sterol dosages and high or moderate omega-3-fatty acid doses. These may have effect on cognitive decline and the onset of Alzheimers disease. The softgel capsules, within the scope of the present invention, allow the optimal delivery of sterols and omega.

### Cardiac Health: Combination of Phytosterols with Tocotrienols

Tocotrienols, particularly gamma tocotrienol, are natural inhibitors of cholesterol synthesis and act by post-transciptional inhibition of HMG CoA reductase (Song.2006). The optimum dose observed for lowering LDL cholesterol in the human was reported to be 100 mg/day (Quershi et al 2002). Tocotrienols, like phytosterols (Mohammed.1997) and omega-3-fatty acids (Erkkila.2004) protect against atheroma formation (Black.2000). Because phytosterols and tocotrienols act by different mechanisms, the effects on LDL cholesterol will be additive. Two examples of phytosterol/tocotrienol combinations are found in the Table below. The softgel capsules, within the scope of the present invention, allow the optimal delivery of sterol, omega and tocotrienols.

### Elevated Stanol Content—Advantages:

TABLE 1
Comparison of The Effects Of Serols, Stanols And Sterol/Stanol
Mixtures On The Blood Levels Of Phytosterols.

| | | | | |
|---|---|---|---|---|
| | | | % Change | |
| | | | in plasma | % Change in |
| | | Dose | B- | plasma |
| Publication | Sterol type | g/day | Sitosterol | Campesterol |

STEROLS

| | | | | |
|---|---|---|---|---|
| Westrate.1998 | Sterol Esters | 3.0 | +39 | +73 |
| Vanstone. 2002 | Sterols | 1.8 | +12 | +72 |
| Ketomaki.2003 | Sterol Esters | 2.0 | +43 | +53 |
| Amundsen.2004 | Sterol Esters | 1.2 | +33 | +76 |

STEROL/STANOL MIXTURES

| | | | | |
|---|---|---|---|---|
| Jones.1999 | Sterol/ | 1.8 | −28 | +4 |
| | Stanol mixture | | | |
| Vanstone. 2002 | Sterol/ | 1.8 | +3 | +28 |
| | Stanol mixture | | | |

STANOLS

| | | | | |
|---|---|---|---|---|
| Gylling.1995a | Stanol Esters | 3.0 | −29 | −42 |
| Westrate.1998 | Stanol Esters | 3.0 | −36 | −17 |
| Vanstone. 2002 | Stanols | 1.8 | −48 | −51 |
| Ketomaki.2003 | Stanol Esters | 2.0 | −32 | −41 |

Changing blood levels of endogenous phytosterols in the blood may have negative effects on health. Blood levels and intakes of phytosterols are higher in individuals eating a vegetarian diet. This type of diet is associated with a lower risk of heart disease. Low doses of tall oil sterols (sitosterol) has been shown to be effective in reducing the symptoms of benign prostate hypertrophy (Berges.2000; Coleman.2002), a condition that affects the majority of males over the age of 40. There are individual differences in the degree to which phytosterols are absorbed from the diet. There is a positive correlation between blood levels of phytosterols and the development of atherosclerosis (Glueck.1991) and coronary events (Assmann.2006). Although it is not certain that elevated phytosterols contribute to these correlations (Sudhop.2002), it would appear to be an advantage to maintain normal blood levels of phytosterols. Mixtures of tall oil sterols and tall oil stanols are neutral in their effects on blood levels of sitosterol and campesterol with effects intermediate to that of stanols or sterols alone. The relative amounts of sterols or stanols required depends on the source of the sterols.

Within the scope of the present invention, it is preferred that the free sterol/stanol component comprise from 10 to 25 wt % free stanols, more preferably from 15-20 wt % (the remainder, of course, being free sterols). Tall oil sterols, as extracted from the source, generally have an endogenous content of stanols of about 9 to 12% (stanols are usually undetectable in vegetable oil sterols). As such, it is preferred that stanols be spiked or blended into the endogenous, extracted composition in order to achieve the preferred, higher concentration of stanols.

There are other advantages of including higher levels of free stanols. Some clinical studies point to a reduction of secretion of bile acids over the long term with sterol-only products resulting in some loss of efficacy in LDL lowering. This effect is thought to result from elevated blood levels of phytosterols. The stanol products are reported to not lose efficacy over the long term (O'Neill et al 2005). In Tables 2,4,6,8,10 formulations with variations in stanol content from 11.6 to 35% are presented.

### Presence of Free Stanols or Sterols

Combining free stanols and/or free sterols with their esterified counterparts, in accordance with the present invention, has the advantage of stabilizing vegetable fats and omega-fatty acids. Omega-fatty acids and likely also tocotrienols are susceptible to degradation by oxidation. It has been found that the presence of free sterols and/or stanols stabilize not only the ester content but the omega fatty acid content of capsules thereby extending shelf-life. Addition of free sterols and/or stanols to the capsules has another key advantage: the capsule volume is reduced. This is not an insignificant feature as it allows the manufacture of smaller capsules better suited for smaller individuals, those with difficulty swallowing, or the elderly. It also allows the flexibility of space within the capsule to add additional, therapeutically beneficial components.

In a most preferred form, to avoid the problem of Ostwald Ripening which is attendant in the use of high concentrations of free sterols/stanols in softgel capsules, it is preferred that the amount of free sterols/stanol be below 40% wt of the fill weight of the capsule. It is preferred, although not required, that the amount of ester be above 20% of the fill weight of the capsule. Exceptions to this include capsules for use in treating prostate disorders, as shown more fully in the examples below.

### Vegetable Oil

It is preferred that vegetable oil is used as the diluent for the ester. Depending on the amount of (and if) free sterols or stanols are also present, the reduced viscosity of the mixture may result in non-uniformity in the fill mixture. A higher viscosity vegetable oil, such as olive oil can be used in this situation. It is fully within the purview of a skilled person in this area to select oils which address such viscosity issues. Tables 10 and 11 present examples of sterol only formulations.

### Prostate Health: Combination of Phytosterols with Omega-3-Fatty Acids

Tall oil phytosterols (β-sitosterol) have been demonstrated to reduce the symptoms of benign prostate hypertrophy (Berges.2000; Coleman.2002). The effective dose of sitosterol is in the range of 20 to 60 mg per day. Both phytosterols (Awad.2000) and omega-3-fatty acids have been observed to either reduce the risk and/or retard the development of prostate cancer (Awad.2002; Augustsson.2003). Examples of dosage forms designed for prostate health are shown in Table 9.

### Softgel Capsules Formation:

The creation and commercial manufacture of softgel capsules is well know in the art, and is not described in detail herein. The formulations and pharmaceutical compositions can be prepared by these known, conventional techniques. Additional, pharmaceutically available excipients and additives can be included, as desired. Such pharmaceutically acceptable excipients and additives include non-toxic compatible fillers, binders, disintegrants, buffers, preservatives, anti-oxidants, lubricants, flavourings, thickeners, colouring agents, emulsifiers and the like.

Within the scope of the present invention, the capsules include push-fit capsules made of, for example, gelatin, as well as soft, sealed capsules made of gelatin and a plasticizer, such as glycerol or sorbitol. In addition, stabilizers may be added. Non-animal gelatin equivalents may be substituted.

### EXAMPLES

The following examples are provided for the purpose of illustration of the invention and not limitation of the invention. A skilled person may use these for guidance in creating the desired softgel capsule formulation, in accordance with the present invention:

### Example 1—Softgel Capsule Formation-Dosages of 0.06-2.00 g/Sterols/Day

A number of considerations need to be taken into account when manufacturing capsules. One is capsule size. The upper size limit which most consumers find acceptable is about 1.20 g, a size commonly used for fish oil supplements. The second consideration is the number of capsules needed to be taken per day. As a matter of practicality, the upper limit is 4 to 6 capsules per day. At the larger capsule size, an oblong shape (1000 mg or more) is preferred as this is easier to swallow. Of course, the present invention is not limited by these parameters.

Phytosterols: The number of portions per day of phytosterol containing products is regulated in the United States and in the European Union. The FDA health claim for phytosterols, requires two servings per day (FDA#1). The European Union requires that phytosterols be dosed in either one portion per day or three portions per day but not two portions (EU labelling regulation). Another consideration is the minimum dose needed for efficacy. The FDA has set this dose at 0.8 g/day. The European Union has not set a minimum dose, but has set a maximum allowable dose of 3.0 g/day. Studies done with the applicant's proprietary tall oil sterols show that a near maximal lowering of LDL occurs at a dose of 1.8 g/day when taken with meals three times a day. The preferred dosage for lowering LDL cholesterol is 1.8 g per day in divided doses.

Omega-3-fatty acids: The FDA recommends that supplement products be labelled to deliver less than 1 g/day (FDA #2). The FDA recommends an upper limit of 3 g/day for intake from supplement products and foods. The American Heart Association recommends a daily intake of 500-1800 mg/day of omega-3-fatty acids from supplements and fish sources.

### Capsules Containing Sterol Esters Plus Other Components

In the following examples, the capsule fill volumes have been kept below 1200 mg by increasing the number of capsules required per day to provide the indicated dosage. The content of sterol esters have been kept above 20% of the fill weight and the content of free sterols/stanols has been kept below 40% to avoid the problem of re-crystallization of the sterols/stanols below the capsule shell. Exceptions are examples 34 and 35 where the sterol content is low enough to dissolve in the lipid fraction. In some of the examples, where there is a high proportion of the fill as omega-3-fatty acids it may be necessary to add a thickening agent to maintain the free sterols/stanols in suspension during the capsule filling operation. With higher proportions of sterol esters, this is unnecessary.

TABLE 2
High Dose PS Plus High Dose Omegas With Various Stanol Contents

| | Dosage | | % Total PhytoSt. | | | Caps | 100 kg of Capsule Filling | | | |
|---|---|---|---|---|---|---|---|---|---|---|
| | Phyto | Omega- | | As | Caps | Fill | Sterol | Free | Free | Omega |
| Form. | Sterols | 3-FA | As | Sterol | per | Wt. | Esters | Sterols | Stanols | Fatty Ac. |
| No. | g/day | g/day | Stanols | Esters | Day | (g) | (kg) | (kg) | (kg) | (kg) |
| 1 | 2.00 | 2.00 | 11.6 | 100.0 | 6 | 1.111 | 50.0 | 0.00 | 0 | 50.00 |
| 2 | 2.00 | 2.00 | 13.0 | 98.4 | 6 | 1.108 | 49.3 | 0.00 | 0.51 | 50.15 |
| 3 | 2.00 | 2.00 | 15.0 | 96.1 | 6 | 1.103 | 48.4 | 0.00 | 1.26 | 50.37 |
| 4 | 2.00 | 2.00 | 20.0 | 90.2 | 6 | 1.091 | 45.9 | 0.00 | 3.14 | 50.92 |
| 5 | 2.00 | 2.00 | 25.0 | 84.4 | 6 | 1.079 | 43.4 | 0.00 | 5.07 | 51.48 |
| 6 | 2.00 | 2.00 | 30.0 | 78.6 | 6 | 1.067 | 40.9 | 0.00 | 7.04 | 52.05 |
| 7 | 2.00 | 2.00 | 35.0 | 72.8 | 6 | 1.055 | 38.3 | 0.00 | 9.04 | 52.64 |

TABLE 3
High Dose PS Plus High Dose Omegas With Various Free Sterol Contents

| | Dosage | | % Total PhytoSt. | | | Caps | 100 kg of Capsule Filling | | | |
|---|---|---|---|---|---|---|---|---|---|---|
| | Phyto | Omega- | | As | Caps | Fill | Sterol | Free | Free | Omega |
| Form. | Sterols | 3-FA | As | Sterol | per | Wt. | Esters | Sterols | Stanols | Fatty Ac. |
| No. | g/day | g/day | Stanols | Esters | Day | (g) | (kg) | (kg) | (kg) | (kg) |
| 8 | 2.00 | 2.00 | 20.0 | 70.2 | 6 | 1.050 | 37.2 | 6.68 | 3.26 | 52.90 |
| 9 | 2.00 | 2.00 | 20.0 | 50.2 | 6 | 1.009 | 27.6 | 13.91 | 3.40 | 55.05 |
| 10 | 2.00 | 2.00 | 20.0 | 30.2 | 6 | 0.968 | 17.3 | 21.74 | 3.54 | 57.37 |

TABLE 4
High Dose PS Plus Medium Dose Omegas With Various Stanol Contents

| | | | % Total | | | | | | | |
|---|---|---|---|---|---|---|---|---|---|---|
| | Dosage | | PhytoSt. | | | Caps | 100 kg of Capsule Filling | | | |
| | Phyto | Omega- | | As | Caps | Fill | Sterol | Free | Free | Omega |
| Form. | Sterols | 3-FA | As | Sterol | per | Wt. | Esters | Sterols | Stanols | Fatty Ac. |
| No. | g/day | g/day | Stanols | Esters | Day | (g) | (kg) | (kg) | (kg) | (kg) |
| 11 | 2.00 | 1.00 | 11.6 | 100.0 | 6 | 0.833 | 66.7 | 0.00 | 0.00 | 33.33 |
| 12 | 2.00 | 1.00 | 13.0 | 98.4 | 6 | 0.830 | 65.9 | 0.00 | 0.68 | 33.46 |
| 13 | 2.00 | 1.00 | 15.0 | 96.1 | 6 | 0.825 | 64.7 | 0.00 | 1.68 | 33.66 |
| 14 | 2.00 | 1.00 | 20.0 | 90.2 | 6 | 0.813 | 61.6 | 0.00 | 4.21 | 34.15 |
| 15 | 2.00 | 1.00 | 25.0 | 84.4 | 6 | 0.801 | 58.5 | 0.00 | 6.83 | 34.66 |
| 16 | 2.00 | 1.00 | 30.0 | 78.6 | 6 | 0.790 | 55.3 | 0.00 | 9.51 | 35.18 |
| 17 | 2.00 | 1.00 | 35.0 | 72.8 | 6 | 0.778 | 52.0 | 0.00 | 12.27 | 35.72 |

TABLE 5
High Dose PS Plus Medium Dose Omegas With Various Free Sterol Contents

| | | | % Total | | | | | | | |
|---|---|---|---|---|---|---|---|---|---|---|
| | Dosage | | PhytoSt. | | | Caps | 100 kg of Capsule Filling | | | |
| | Phyto | Omega- | | As | Caps | Fill | Sterol | Free | Free | Omega |
| Form. | Sterols | 3-FA | As | Sterol | per | Wt. | Esters | Sterols | Stanols | Fatty Ac. |
| No. | g/day | g/day | Stanols | Esters | Day | (g) | (kg) | (kg) | (kg) | (kg) |
| 18 | 2.00 | 1.00 | 20.0 | 70.2 | 6 | 0.772 | 50.5 | 9.09 | 4.44 | 35.96 |
| 19 | 2.00 | 1.00 | 20.0 | 50.2 | 4 | 1.097 | 38.2 | 19.19 | 4.69 | 37.98 |
| 20 | 2.00 | 1.00 | 20.0 | 30.2 | 4 | 1.036 | 24.3 | 30.49 | 4.96 | 40.23 |

TABLE 6
High Dose PS Plus Low Dose Omegas With Various Stanol Contents

| | | | % Total | | | | | | | |
|---|---|---|---|---|---|---|---|---|---|---|
| | Dosage | | PhytoSt. | | | Caps | 100 kg of Capsule Filling | | | |
| | Phyto | Omega- | | As | Caps | Fill | Sterol | Free | Free | Omega |
| Form. | Sterols | 3-FA | As | Sterol | per | Wt. | Esters | Sterols | Stanols | Fatty Ac. |
| No. | g/day | g/day | Stanols | Esters | Day | (g) | (kg) | (kg) | (kg) | (kg) |
| 21 | 2.00 | 0.50 | 11.6 | 100.0 | 4 | 1.042 | 80.0 | 0.00 | 0.00 | 20.00 |
| 22 | 2.00 | 0.50 | 13.0 | 98.4 | 4 | 1.037 | 79.1 | 0.00 | 0.81 | 20.09 |
| 23 | 2.00 | 0.50 | 15.0 | 96.1 | 4 | 1.030 | 77.7 | 0.00 | 2.02 | 20.24 |
| 24 | 2.00 | 0.50 | 20.0 | 90.2 | 4 | 1.012 | 74.3 | 0.00 | 5.08 | 20.59 |
| 25 | 2.00 | 0.50 | 25.0 | 84.4 | 4 | 0.994 | 70.8 | 0.00 | 8.26 | 20.96 |
| 26 | 2.00 | 0.50 | 30.0 | 78.6 | 4 | 0.976 | 67.1 | 0.00 | 11.54 | 21.35 |
| 27 | 2.00 | 0.50 | 35.0 | 72.8 | 4 | 0.958 | 63.3 | 0.00 | 14.94 | 21.74 |

TABLE 7
High Dose PS Plus Low Dose Omegas With Various Free Sterol Contents

| | Dosage | | % Total PhytoSt. | | | Caps | 100 kg of Capsule Filling | | | | |
|---|---|---|---|---|---|---|---|---|---|---|---|
| | Phyto | Omega- | | As | Caps | Fill | Sterol | Free | Free | Omega | Edible |
| Form. | Sterols | 3-FA | As | Sterol | per | Wt. | Esters | Sterols | Stanols | Fatty Ac. | Oil |
| No. | g/day | g/day | Stanols | Esters | Day | (g) | (kg) | (kg) | (kg) | (kg) | (kg) |
| 28 | 2.00 | 0.50 | 20.0 | 70.2 | 4 | 0.950 | 61.6 | 11.08 | 5.41 | 21.92 | 0.000 |
| 29 | 2.00 | 0.50 | 20.0 | 50.2 | 4 | 0.889 | 47.1 | 23.68 | 5.79 | 23.44 | 0.000 |
| 30 | 2.00 | 0.50 | 20.0 | 40.2 | 4 | 0.858 | 39.1 | 30.67 | 5.99 | 24.28 | 0.000 |

TABLE 8
Medium Dose PS Plus Medium Dose Omegas With Variable Stanol Content

| | | | % Total | | | | | | | |
|---|---|---|---|---|---|---|---|---|---|---|
| | Dosage | | PhytoSt. | | | Caps | 100 kg of Capsule Filling | | | |
| | Phyto | Omega- | | As | Caps | Fill | Sterol | Free | Free | Omega |
| Form. | Sterols | 3-FA | As | Sterol | per | Wt. | Esters | Sterols | Stanols | Fatty Ac. |
| No. | g/day | g/day | Stanols | Esters | Day | (g) | (kg) | (kg) | (kg) | (kg) |
| 31 | 1.00 | 1.00 | 15.0 | 36.1 | 4 | 0.735 | 20.4 | 21.48 | 1.41 | 56.68 |
| 32 | 1.00 | 1.00 | 20.0 | 30.2 | 4 | 0.726 | 17.3 | 21.74 | 3.54 | 57.37 |
| 33 | 1.00 | 1.00 | 25.0 | 24.4 | 4 | 0.717 | 14.2 | 22.01 | 5.72 | 58.09 |

TABLE 9
Very Low Dose PS Combined With Moderate & High Dose Omegas

| | | | % Total | | | | | | | |
|---|---|---|---|---|---|---|---|---|---|---|
| | Dosage | | PhytoSt. | | | Caps | 100 kg of Capsule Filling | | | |
| | Phyto | Omega- | | As | Caps | Fill | Sterol | Free | Free | Omega |
| Form. | Sterols | 3-FA | As | Sterol | per | Wt. | Esters | Sterols | Stanols | Fatty Ac. |
| No. | g/day | g/day | Stanols | Esters | Day | (g) | (kg) | (kg) | (kg) | (kg) |
| 34 | 0.06 | 1.00 | 11.6 | 40.0 | 2 | 0.872 | 2.3 | 2.17 | 0.00 | 95.54 |
| 35 | 0.06 | 1.80 | 11.6 | 40.0 | 3 | 1.026 | 1.3 | 1.23 | 0.00 | 97.47 |

TABLE 10
High Dose PS With Various Stanol Contents in Vegetable Oil

| | Dosage | | % Total PhytoSt. | | | Caps | 100 kg of Capsule Filling | | | | |
|---|---|---|---|---|---|---|---|---|---|---|---|
| | Phyto | Omega- | | As | Caps | Fill | Sterol | Free | Free | Omega | Olive |
| Form. | Sterols | 3-FA | As | Sterol | per | Wt. | Esters | Sterols | Stanols | Fatty Ac. | Oil |
| No. | g/day | g/day | Stanols | Esters | Day | (g) | (kg) | (kg) | (kg) | (kg) | (kg) |
| 36 | 2.00 | 0.00 | 11.6 | 100.0 | 4 | 0.948 | 87.9 | 0.00 | 0.00 | 0.00 | 12.09 |
| 37 | 2.00 | 0.00 | 13.0 | 98.4 | 4 | 0.943 | 87.0 | 0.00 | 0.89 | 0.00 | 12.15 |
| 38 | 2.00 | 0.00 | 15.0 | 96.1 | 4 | 0.936 | 85.5 | 0.00 | 2.22 | 0.00 | 12.24 |
| 39 | 2.00 | 0.00 | 20.0 | 90.2 | 4 | 0.918 | 81.9 | 0.00 | 5.60 | 0.00 | 12.48 |
| 40 | 2.00 | 0.00 | 25.0 | 84.4 | 4 | 0.900 | 78.1 | 0.00 | 9.12 | 0.00 | 12.73 |
| 41 | 2.00 | 0.00 | 30.0 | 78.6 | 4 | 0.882 | 74.2 | 0.00 | 12.77 | 0.00 | 12.99 |
| 42 | 2.00 | 0.00 | 35.0 | 72.8 | 4 | 0.864 | 70.2 | 0.00 | 16.56 | 0.00 | 13.26 |

TABLE 11
High Dose PS With Various Sterol Contents in Vegetable Oil

| | Dosage | | % Total PhytoSt. | | | Caps | 100 kg of Capsule Filling | | | | |
|---|---|---|---|---|---|---|---|---|---|---|---|
| | Phyto | Omega- | | As | Caps | Fill | Sterol | Free | Free | Omega | Olive |
| Form. | Sterols | 3-FA | As | Sterol | per | Wt. | Esters | Sterols | Stanols | Fatty Ac. | Oil |
| No. | g/day | g/day | Stanols | Esters | Day | (g) | (kg) | (kg) | (kg) | (kg) | (kg) |
| 43 | 2.00 | 0.00 | 20.0 | 90.2 | 4 | 0.918 | 81.9 | 0.00 | 5.60 | 0.00 | 12.48 |
| 44 | 2.00 | 0.00 | 20.0 | 70.2 | 4 | 0.909 | 64.4 | 11.59 | 5.66 | 0.00 | 18.34 |
| 45 | 2.00 | 0.00 | 20.0 | 50.2 | 4 | 0.914 | 45.8 | 23.04 | 5.63 | 0.00 | 25.53 |
| 46 | 2.00 | 0.00 | 20.0 | 30.2 | 4 | 0.883 | 38.0 | 29.98 | 5.82 | 0.00 | 26.42 |

TABLE 12
High Dose PS Plus 100 mg/day Tocotrienols In Rice Bran Oil Without or
With Omega-3-Fatty Acids

| | Dosage | | % Total PhytoSt. | | | Caps | 100 kg of Capsule Filling | | | | |
|---|---|---|---|---|---|---|---|---|---|---|---|
| | Phyto | Omega- | | As | Caps | Fill | Sterol | Free | Free | Omega | Edible |
| Form. | Sterols | 3-FA | As | Sterol | per | Wt. | Esters | Sterols | Stanols | Fatty Ac. | Oil* |
| No. | g/day | g/day | Stanols | Esters | Day | (g) | (kg) | (kg) | (kg) | (kg) | (kg) |
| 47 | 2.00 | 0.00 | 20.0 | 70.2 | 4 | 0.867 | 67.5 | 12.14 | 5.93 | 0.00 | 14.42 |
| 48 | 2.00 | 0.50 | 20.0 | 70.2 | 4 | 1.075 | 54.4 | 9.79 | 4.78 | 19.38 | 11.63 |

*15% tocotrienol (primarily gamma-tocotrienol) in rice bran oil; daily dose of tocotrienols, 100 mg.

The relative proportions of sterols and omegas depends on the market. If the consumer views the omega-3-fatty acid content as the more important parameter, formulations as shown in Tables 2, 3 and 9 might be preferred. The cholesterol reduction claim for sterols is much stronger in the United States; it is a full claim. The omega fatty acid health claim is only a qualified claim and must include the statement “Supportive but not conclusive research shows that consumption of EPA and DHA omega-3-fatty acids may reduce the risk of heart disease”. If the phytosterol component is to be emphasized, there are many formulations clearly showing this preference. If the objective is maximum efficacy of both components, other formulations clearly show this.

The fish source omega-fatty acids have two active components, DHA and EPA. These have different activities. For example, DHA is effective in preventing certain brain disorders. Fish oil omega fatty acids are available with different ratios of DHA and EPA, these generally have a purity of about 60%. The type of oil used can be varied depending on the purpose of the supplement. Such is within the purview of a skilled person in this field.

### Example 2—Softgel Capsule Formation-Dosages of 0.06-1.8 g/Sterols/Day

TABLE 13
Formulations:

| | | Omega | Percent | Percent | | | 100 kg Batch | | |
|---|---|---|---|---|---|---|---|---|---|
| | | Fatty | Sterols | Sterols | Capsules | Capsule | | Omega | |
| | Sterols | Acids | as | as | per | Fill Wt. | Sterol | Fatty | Tall Oil |
| Example# | g/day | g/day | Stanols | Esters | Day | Gm | Esters | Acids | Stanols |

Cardiovascular Disease, Cancer Risk Reduction, Neurodegenerative diseases

| | | | | | | | | | |
|---|---|---|---|---|---|---|---|---|---|
| 1 | 1.800 | 0.500 | 11.6 | 100 | 4 | 0.958 | 78.26 | 21.74 | 0 |
| 2 | 1.800 | 0.500 | 11.6 | 100 | 6 | 0.639 | 78.26 | 21.74 | 0 |
| 3 | 1.800 | 0.900 | 11.6 | 100 | 4 | 1.125 | 66.67 | 33.33 | 0 |
| 4 | 1.800 | 0.900 | 11.6 | 100 | 6 | 0.750 | 66.67 | 33.33 | 0 |
| 5 | 1.800 | 1.800 | 11.6 | 100 | 6 | 1.000 | 50.00 | 50.00 | 0 |
| 6 | 0.900 | 1.800 | 11.6 | 100 | 4 | 1.125 | 33.33 | 66.67 | 0 |
| 7 | 0.900 | 1.800 | 11.6 | 100 | 6 | 0.750 | 33.33 | 66.67 | 0 |

Benign Prostate Hyperplasia Indication

| | | | | | | | | | |
|---|---|---|---|---|---|---|---|---|---|
| 8 | 0.060 | 1.800 | 11.6 | 100 | 3 | 1.033 | 3.23 | 96.77 | 0 |
| 9 | 0.060 | 1.800 | 11.6 | 100 | 4 | 0.775 | 3.23 | 96.77 | 0 |

Examples where stanol content of sterol mixture has been increased.

| | | | | | | | | | |
|---|---|---|---|---|---|---|---|---|---|
| 10 | 1.80 | 0.90 | 14.00 | 97.21 | 4 | 1.117 | 65.25 | 33.56 | 1.183 |
| 11 | 1.80 | 0.90 | 14.00 | 97.21 | 6 | 0.745 | 65.25 | 33.56 | 1.183 |
| 12 | 1.80 | 0.90 | 20.00 | 90.23 | 4 | 1.098 | 61.63 | 34.15 | 4.21 |
| 13 | 1.80 | 0.90 | 20.00 | 90.23 | 6 | 0.732 | 61.63 | 34.15 | 4.21 |
| 14 | 1.80 | 1.80 | 20.00 | 90.23 | 6 | 0.982 | 45.94 | 50.92 | 3.14 |
| 15 | 0.90 | 1.80 | 30.00 | 78.60 | 4 | 1.095 | 26.91 | 68.47 | 4.63 |
| 16 | 0.90 | 1.80 | 30.00 | 78.60 | 6 | 0.730 | 26.91 | 68.47 | 4.63 |

Formulations 1-7 would be useful to reduce the risk of cancer, Alzheimer's disease or other neurodegenerative diseases. The preferred mixture would be formulation 5.

Formulations 8 and 9 are intended for use in ameliorating the symptoms of benign prostate hypertrophy. The effective doses of sitosterol (tall oil sterols) are 20 to 60 mg per day.

Formulations 10-16 are essentially the same as formulations 3 and 4 except that the stanol content of the capsules has been increased from that typical of tall oil sterols (11.6%) to 16%, 18%, and 20%. Increasing the tall stanol content slightly reduces the volume of the fill because the amount of ester required to fill out the total dose of phytosterols is reduced.

Some of the preferred formulations shown relate primarily to the prevention of cardiovascular disease. A cholesterol reduction claim is allowed in the United States. The EU labelling regulation allows use of the statement “this product is intended only for persons who want to lower their blood cholesterol”. In the some of the examples provided, dosages of 4 and 6 capsules per day are used. The health claim allows instructions based on two servings per day. The EU labelling regulation requires that dosing instructions be based on either one serving per day or three servings per day but not two. The 4 per day examples can be used in the United States. For example, take two capsules twice a day. The 6 capsules per day examples can be used in either area. For example, in the U.S., take 3 capsules twice a day. In the EU the instruction would be take 2 capsules three times per day.

Formulation number 10 was tested on commercial scale encapsulation equipment with very good results. On a 50 kg batch, capsule recoveries were 95%.

### Example 3—Formulation No. 10 (Table 13)

Capsule ingredients:
1. EPAX6000EE (Pronova) fish source omega fatty acids with a purity of 60%.
2. Tall oil sterol esters with a sterol content of 60% by weight and a stanol concentration in the sterols of 11.6%.
3. Tall oil stanols with a purity of 97.6%.
4. Gelatin: beef gelatin (BSE free), glycerin, water, titanium dioxide masking agent, and a light yellow colouring agent.

### Procedure:

The fish oil was poured into a mixing vessel and stanols added. The stanols dissolved almost immediately in omega fatty acid oil. The indicated amount of sterol esters were warmed to point where the indicated amount of could be added to the mixing vessel. The mixture was then encapsuled with a beef gelatin (BSE free) containing glycerin, water, titanium dioxide masking agent and a light yellow colouring agent. The capsule shape used was oblong. The capsules were dried for two days at room temperature before packaging.

Capsules can be either animal based gelatin (beef, pork, or fish) or vegetable based gelatin. Colour and shape are optional. An off-odour sometimes comes from the animal gelatins or the fish oil. Vanillin has been found to be a good odour masking agent when incorporated into the gelatin.

### Example 4—(Dissolution of Stanols in Esters)

Softgel capsule Filler Composition of:
0.344% stanol
11.5% sterol esters

### Stanols:

### Code: FCP-3P2

### BRI ID: FM-P2-83

### Manuf.lot# 5QC27H-2

### Description (dry form, room temp):

white powder composed of longitudinally shaped granules/crystals. White flour-like feel.

### Sterol Esters:

### Description:

### Temp (−) 18C—solid

### Temp 13-15C—sticky texture, viscosity less than that of ,for example, honey at room temperature, therefore has a semi-high viscosity . Able to be pumped?—no

### Temp 35C-viscosity—slightly more viscous than regular canola oil at room temp—

appearance—resembles canola oil (clear, golden color)

### Temp 60C viscosity—that of canola oil at room temp, however more sticky to touch than normal oil

### Stanols with Sterol Esters Description

1. 0.344 g stanol added to 11.5 g sterol esters. Sterol ester increased to temp 90° C.

### Stanols dissolved fully

2. Stanol/sterol esters, fully dissolved, cooled to 35C
   No apparent recrystallization of stanols into its original crystals. Sterol esters appear smooth , no appearance of crystals within
3. Stanol/sterol esters, same sample as above, cooled to 25C

### Stanols dissolved fully, Good appearance and handling.

### End Composition: 97% esters and 3% stanols

### REFERENCES

1. Law M. R., Wald N. J., Wu., Hacksaw Z A., Bailey A.; Systemic underestimation of association between serum cholesterol concentration and ischemic heart disease in observational studies: Data from BUPA Study; Br. Med. J. 1994; 308:363-366
2. Law M. R., Wald N. J., Thompson S. G.; By how much and how quickly does reduction in serum cholesterol concentration lower risk of ischemic heart disease? Br. Med. J. 1994; 308:367-373
3. Kannel W B, Castelli W P, Gordon T et al. Lipoprotein cholesterol in the prediction of atherosclerotic disease: new perspectives based on the Framingham Heart Study. Ann Intern Med. 1995;90:85-91
4. Singh B K, Mehta J L. Management of dyslipidemia in the primary prevention of coronary heart disease. Curr Opin Cardiol. 2002; 17:503-11
5. La Rosa J. C., Hunninghake D. Bush D. et al.; The cholesterol facts: A summary of the evidence relating to dietary fats, serum cholesterol and coronary heart disease:A joint statement by the American Heart Association and the National Heart, Lung and Blood Institute. Circulation 1990; 81:1721-1733
6. Havel R. J., Rapaport E. Drug Therapy: Management of Primary Hyperlipidemia. New England Journal of Medicine, 1995; 332:1491-1498
7. Kuccodkar et al.; Effects of plant sterols on cholesterol metabolism. Atherosclerosis, 1976; 23:239-248
8. Lees R. S., Lees A. M. Effects of sitosterol therapy on plasma lipid and lipoprotein concentrations. In: Greten H (Ed) Lipoprotein Metabolism. Springer-Verlag, Berlin, Heidelberg, New York, 1976:119-124
9. Lees A. M., Mok H. Y. I., Lees R. S., McCluskey M. A., Grundy S. M. Plant sterols as cholesterol-lowering agents: clinical trials in patients with hypercholesterolemia and studies of sterol balance. Atherosclerosis 1977; 28: 325-338

### ADDITIONAL REFERENCES

Assmann G, Cullen P, Erbey J, Ramey D R, Kannenberg F, Schulte H. Plasma sitosterol elevations are associated with an increased incidence of coronary events in men: results of a nested case-control analysis of the Prospective Cardiovascular Munster (PROCAM) study. Nutr Metab Cardiovasc Dis. 2006 January; 16(1):13-21.
Aviram M, Kassem E. 1993. Dietary olive oil reduces low-density lipoprotein uptake by macrophages and decreases the susceptibility of the lipoprotein to undergo lipid peroxidation. Ann Nutr Metab 37:75-84.
Augustsson K, Michaud D S, Rimm E B, Leitzmann M F, Stampfer M J, Willett W C, Giovannucci E. A prospective study of intake of fish and marine fatty acids and prostate cancer. Cancer Epidemiol Biomarkers Prev. 2003 January; 12(1):64-7.
Awad A B, Fink C S. Phytosterols as anticancer dietary components: evidence and mechanism of action. J. Nutr. 2000 September; 130(9):2127-30.
Awad A B, Fink C S, Williams H, Kim U. In vitro and in vivo (SCID mice) effects of phytosterols on the growth and dissemination of human prostate cancer PC-3 cells. Eur J Cancer Prev. 2001 December; 10(6):507-13.
Bartsch H, Nair J, Owen R W. Dietary polyunsaturated fatty acids and cancers of the breast and colorectum: emerging evidence for their role as risk modifiers. Carcinogenesis. 1999 December; 20(12):2209-18.
Berges R R, Kassen A, Senge T. Treatment of symptomatic benign prostatic hyperplasia with beta-sitosterol: an 18-month follow-up. BJU Int. 2000 May; 85(7):842-6.
Black T M, Wang P, Maeda N, Coleman R A. Palm tocotrienols protect ApoE +/−mice from diet-induced atheroma formation. J. Nutr. 2000 October; 130(10):2420-6.
Bourque C, St-Onge M P, Papamandjaris A A, Cohn J S, Jones P J. Consumption of an oil composed of medium chain triacyglycerols, phytosterols, and N-3 fatty acids improves cardiovascular risk profile in overweight women. Metabolism. 2003 June; 52(6):771-7.
Caygill C P, Charlett A, Hill M J. Fat, fish, fish oil and cancer. Br J Cancer. 1996 July; 74(1):159-64.
Coleman C I, Hebert J H, Reddy P. The effect of phytosterols on quality of life in the treatment of benign prostatic hyperplasia. Pharmacotherapy. 2002 November; 22(11):1426-32.
Erkkila A T, Lichtenstein A H, Mozaffarian D, Herrington D M. Fish intake is associated with a reduced progression of coronary artery atherosclerosis in postmenopausal women with coronary artery disease. Am J Clin Nutr. 2004 September; 80(3):626-32.
FDA Letter to Emord & Associates, P. C., 1050 17^{th} Street, N.W. Suite 600, Washington, D.C. 20036 from U.S. Food and Drug Administration, Center for Food Safety and Applied Nutrition, Office of Nutritional Products, Labeling, and Dietary Supplements, Oct. 31, 2000.
Frenoux J M, Prost E D, Belleville J L, Prost J L(2001) A polyunsaturated fatty acid diet lowers blood pressure and improves antioxidant status in spontaneously hypertensive rats. J Nutr 131(11):39-45.
Glueck C J, Speirs J, Tracy T, Streicher P, Illig E, Vandegrift J. Relationships of serum plant sterols (phytosterols) and cholesterol in 595 hypercholesterolemic subjects, and familial aggregation of phytosterols, cholesterol, and premature coronary heart disease in hyperphytosterolemic probands and their first-degree relatives. Metabolism. 1991 August; 40(8):842-8.
Grundy S M. 1990. Cholesterol and atherosclerosis: diagnosis and treatment. ISBN 0-397-44674-8.
Gupta M B, Nath R, Srivastava N, Shanker K, Kishor K, Bhargava K P. 1980. Anti-inflammatory and antipyretic activities of beta-sitosterol. Planat Medica 39:157-163.
Hagiwara H, Shimonaka M, Morisaki M, Ikekawa N, Inada Y. 1984. Sitosterol-stimulative production of plasminogen activator in cultured endothelial cells from bovine carotid artery. Thromb Research 33:363-70.
Harris, W S (1996) n-3 Fatty acids and lipoproteins: Comparison of results from human and animal studies. Lipids 31:243-252.
Hoffmann A, Klocking H P. 1988. Influence of beta-sitosterol on the fibrinolytic potential in rabbits. Folia Haematol Leipzig 1-2:189-96.
Jones P J, Ntanios F Y, Raeini-Sarjaz M, Vanstone C A. Cholesterol-lowering efficacy of a sitostanol-containing phytosterol mixture with a prudent diet in hyperlipidemic men. Am J Clin Nutr 1999 June; 69(6):1144-50
Katan, M B, Grundy S M, Jones P, Law M, Miettinen T, Paoletti R. Efficacy and safety of plant stanols and sterols in the management of blood cholesterol levels. Mayo Clin Proc 78:965-978, 2003.
Krauss R M. Hold the antioxidants and improve plasma lipids? J Clin Invest. 2004 May; 1 3(9):1253-5.
Kris-Etherton P M, Harris W S, Appel L J. AHA Statement. Fish consumption. Fish Oil. Omega-3 fatty acids, and cardiovascular disease. Circulation 2002 106:2747-2757.
Luchsinger J A, Mayeux R. Dietary factors and Alzheimer's disease. Lancet Neurol. 2004 October; 3(10):579-87.
Marchioli R, et al. Early protection against sudden death by n-3 polyunsaturated fatty acids after myocardial infarction: time-course analysis of the results of the Gruppo Italiano per lo Studio della Sopravvivenza nell'lnfarto Miocarico (GISSI-Prevenzione. Circulation 2002 105:1897-1903.
Maroon J C, Bost J W. Omega-3 fatty acids (fish oil) as an anti-inflammatory: an alternative to nonsteroidal anti-inflammatory drugs for discogenic pain. Surg Neurol. 2006 April; 65(4):326-31.
Moghadasian M H, McManus B M, Pritchard P H, Frohlich J J. 1996. “Tall oil”—Derived phytosterols reduce atherosclerosis in apoE-deficient mice. Arter Thromb Vasc Biol 17:119-26.
Mori Y, Nobukata H, Harada T, Kasahara T, Tajima N. Long-term administration of highly purified eicosapentaenoic acid ethyl ester improves blood coagulation abnormalities and dysfunction of vascular endothelial cells in Otsuka Long-Evans Tokushima fatty rats. Endocr J. 2003 October; 50(5):603-11.
Mozaffarian D, Psaty B M, Rimm E B, Lemaitre R N, Burke G L, Lyles M F, Lefkowitz D, Siscovick D S. Fish intake and risk of incident atrial fibrillation. Circulation. 2004 Jul. 27; 110(4):368-73.
Mozaffarian D, Longstreth W T Jr, Lemaitre R N, Manolio T A, Kuller L H, Burke G L, Siscovick D S. Fish consumption and stroke risk in elderly individuals: the cardiovascular health study. Arch Intern Med. 2005 January 24; 165(2):200-6.
Nordoy A, Marchioli R, Arnesen H, Videbaek J. n-3 polyunsaturated fatty acids and cardiovascular diseases. Lipids 2001; 36 Suppl:S127-9
Nordoy A, Bonaa K H, Sandset P M, Hansen J B, Nilsen H. Effect of omega-3 fatty acids and simvastatin on hemostatic risk factors and postprandial hyperlipemia in patients with combined hyperlipemia. Arterioscler Thromb Vasc Biol. 2000 January; 20(1):259-65.
O'Neill F H, Sanders T A, Thompson G R. Comparison of efficacy of plant stanol ester and sterol ester: short-term and longer-term studies. Am J Cardiol. 2005 Jul. 4; 96(1A):29D-36D.
Prisco D, Paniccia R, Bandinelli B, Filippini M, Francalanci I, Giusti B, Giurlani L, Gensini G F, Abbate R, Neri Serneri G G. Effect of medium-term supplementation with a moderate dose of n-3 polyunsaturated fatty acids on blood pressure in mild hypertensive patients. Thromb Res 1998 Aug 1 ; 91 (3):105-12
Qureshi A A, Sami S A, Salser W A, Khan F A. Dose-dependent suppression of serum cholesterol by tocotrienol-rich fraction (TRF25) of rice bran in hypercholesterolemic humans. Atherosclerosis. 2002 March; 161(1):199-207.
Renier, G, Skamene, E, DeSanctis, J, Radzioch, D. Dietary n-3 polyunsaturated fatty acids prevent the development of atherosclerotic lesions in mice. Arterioscler. Thrombo. 1993 13:1515-1524,
Roche H M, Gibney M J. Postprandial triacylglycerolaemia: the effect of low-fat dietary treatment with and without fish oil supplementation. Eur J Clin Nutr. 1996 September; 50(9):617-24
Sayeed S A, Farnaz S, Simjee R U, Malik A. 1993. Triterpenes and beta-sitostenol from piper betle: isolation, antiplatelet and anti-inflammatory effects. Bioschem Soc Trans 1993, 21:462S
Schmidt E B, Christensen J H, Aardestrup I, Madsen T, Riahi S, Hansen V E, Skou H A. Marine n-3 fatty acids: basic features and background. Lipids. 2001 ; 36 Suppl:S65-8.
Song B L, DeBose-Boyd R A. Insig-dependent ubiquitination and degradation of 3-hydroxy-3-methylglutaryl coenzyme a reductase stimulated by delta- and gamma-tocotrienols. J Biol. Chem. 2006 Sep 1 ; 281 (35):25054-61. Epub 2006 Jul 10.
Stone N J. The Gruppo Italiano per lo Studio della Sopravvivenza nell'lnfarto Miocardio (GISSI)-Prevenzione Trial on fish oil and vitamin E supplementation in myocardial infarction survivors. Curr Cardiol Rep 2000 September; 2(5):445-51
Sudhop T, Gottwald B M, von Bergmann K. Serum plant sterols as a potential risk factor for coronary heart disease. Metabolism. 2002 December; 51 (12):1519-21.
Theobald H E, Chowienczyk P J, Whittall R, Humphries S E, Sanders T A. LDL cholesterol-raising effect of low-dose docosahexaenoic acid in middle-aged men and women. Am J Clin Nutr. 2004 April; 79(4):558-63.
Vanschoonbeek K, de Maat M P, Heemskerk J W. Fish oil consumption and reduction of arterial disease. J Nutr. 2003 March; 133(3):657-60.
von Schacky C, Baumann K, Angerer P. The effect of n-3 fatty acids on coronary atherosclerosis: results from SCIMO, an angiographic study, background and implications. Lipids. 2001 ;36 Suppl:S99-102.

We claim:

1. A composition for use in softgel capsules comprising one or more esters of phytosterols and/or phytostanols which have been treated to enhance their flowability at ambient temperatures prior to or concurrent with softgel formation.

2. A composition for use in softgel capsules comprising one or more esters of phytosterols and/or phytostanols which have been treated to reduce their viscosity at ambient temperatures.

3. A composition for use in softgel capsules comprising one or more esters of phytosterols and/or phytostanols which have been pre-mixed with an edible oil prior to softgel capsule formation in order to enhance the flowability of the esters at ambient temperatures.

4. A composition for use in softgel capsules comprising one or more esters of phytosterols and/or phytostanols which have been pre-mixed with an edible oil comprising omega polyunsaturated fatty acids prior to softgel capsule formation in order to reduce the viscosity of the esters at ambient temperatures.

5. A method of stabilizing from oxidation a composition of one or more esterified phytosterols and phytostanols such composition being useful for softgel capsule filler, which comprises solubilizing in the esters one or more free (unesterified) phytosterols or phytostanols.

6. The composition of claim 3 wherein the edible oil is selected from the group consisting of olive, rapeseed, canola, sunflower, safflower, sesame, soyabean, corn, coconut, peanut, cottonseed, hemp, flaxseed, and pumpkinseed.

7. The composition of claim 3 wherein the edible oil is high in one or more of omega 3 polyunsaturated fatty acids, omega 6 polyunsaturated fatty acids and omega 9 polyunsaturated fatty acids.

8. The composition of claim 3 wherein the edible oil is of marine or fish origin.

9. The composition of claim 3 supplemented with omega fatty acids derived from microalgae.

10. The composition of claim 1 wherein the phytosterol is selected from the group consisting of sitosterol, campesterol, stigmasterol, brassicasterol (including dihydrobrassicasterol), desmosterol, chalinosterol, poriferasterol, clionasterol, ergosterol, coprosterol, codisterol, isofucosterol, fucosterol, clerosterol, nervisterol, lathosterol, stellasterol, spinasterol, chondrillasterol, peposterol, avenasterol, isoavenasterol, fecosterol, pollinastasterol and all natural or synthesized forms and derivatives thereof, including isomers.

11. The composition of claim 1 wherein the phytostanol is selected from the group consisting of all saturated or hydrogenated phytosterols and all natural or synthesized forms and derivatives thereof, including isomers.

12. The composition claim 1 wherein the esters of phytosterols and/or phytostanols are in form selected from the group consisting of: aliphatic acid esters, aromatic acid esters, phenolic acid esters, cinnamate esters, ferulate esters, phytosterol/phytostanol glycosides, and phytosterol/phytostanol acylglycosides .

13. A softgel capsule comprising one or more esters of phytosterols and/or phytostanols and component to enhance the flowability of the esters.

14. A softgel capsule comprising one or more esters of phytosterols and/or phytostanols and component to reduce the viscosity of the esters at ambient temperatures.

15. A softgel capsule comprising one or more esters of phytosterols and/or phytostanols and a measurable amount of one or more free phytosterols and/or phytostanols.

16. A softgel capsule comprising one or more esters of phytosterols and/or phytostanols and an edible oil.

17. A softgel capsule comprising one or more esters of phytosterols and/or phytostanols a measurable amount of one or more free phytosterols and/or phytostanols and an edible oil.

18. The softgel capsule of claim 16 comprising omega-3 polyunsaturated fatty acids.

19. The softgel capsule of claim 17 comprising omega-3 polyunsaturated fatty acids.
